(19) United States
(12) Patent Application Publication
(10) Pub. No.: US 2024/0017235 A1
(43) Pub. Date: Jan. 18, 2024

(54) Systems, Devices and Methods for Intensification of Reformers and Downstream Chemical Synthesis

(71) Applicant: M2X Energy Inc., Rockledge, FL (US)

(72) Inventors: Paul E. Yelvington, Rockledge, FL (US); Edwin Yik, Orlando, FL (US); John Anthony Dean, Scotia, NY (US); Joshua B. Browne, New York, NY (US)

(73) Assignee: M2X Energy Inc., Rockledge, FL (US)

(21) Appl. No.: 18/198,279

(22) Filed: May 16, 2023

Related U.S. Application Data
(63) Continuation-in-part of application No. PCT/US2022/029707, filed on May 17, 2022.
Application No. PCT/US2022/029707 is a continuation-in-part of application No. 17/746,937, filed on May 17, 2022.
Application No. 17/746,937 is a continuation-in-part of application No. 17/953,056, filed on Sep. 26, 2022.
Application No. 17/953,056 is a continuation-in-part of application No. PCT/US2022/044724, filed on Sep. 26, 2021.
(60) Provisional application No. 63/343,087, filed on May 18, 2022.
(60) Provisional application No. 63/189,756, filed on May 18, 2021.
(60) Provisional application No. 63/197,898, filed on Jun. 7, 2021.
(60) Provisional application No. 63/213,129, filed on Jun. 21, 2021.
(60) Provisional application No. 63/304,463, filed on Jan. 28, 2022.
(60) Provisional application No. 63/248,519, filed on Sep. 26, 2021.

(51) Int. Cl. B01J 19/00; B01J 19/24; B01J 35/04; C07C 29/152; B01D 3/00; B01D 53/02; B01D 53/22

(52) U.S. Cl. CPC B01J 19/0013; B01J 19/0093; B01J 19/2415; B01J 35/04; C07C 29/152; B01D 3/009; B01D 53/02; B01D 53/229

(57) ABSTRACT

Intensified plants comprising chemical reformers and downstream chemical synthesis equipment. Systems, methods and devices for process intensification (PI) that result in lowering part count, merging functionality, removing bottlenecks, reducing costs, and modularizing subsystems for ease of assembly and maintenance to obtain the overall simplification needed to achieve competitive product cost at small scales. In an embodiment, the improved plants employ engine reformers to produce synthesis gas, which is further converted into end products using intensified downstream reactors.

This application: (i) claims under 35 U.S.C. § 119(e)(1) the benefit of the filing date of, and claims the benefit of priority to, U.S. provisional application Ser. No. 63/343,087 filed May 18, 2022; (ii) claims priority to and is a continuation-in-part of PCT patent application serial number PCT/US2022/029707 filed May 17, 2022; and, (iii) claims priority to and is a continuation-in-part of U.S. patent application Ser. No. 17/746,937 filed May 17, 2022, which claims priority to US provisional patent application Ser. No. 63/189,756 filed May 18, 2021, 63/213,129 filed Jun. 17, 2021, 63/197,898 filed Jun. 7, 2021, and 63/304,463 filed Jan. 28, 2022; (iv) claims priority to and is a continuation-in-part of PCT patent application serial number PCT/US2022/044724 filed Sep. 26, 2022; (v) claims priority to and is a continuation-in-part of U.S. patent application Ser. No. 17/953,056 filed Sep. 26, 2022, which claims priority to US provisional patent application Ser. No. 63/248,519 filed Sep. 26, 2021, the entire disclosure of each of which is incorporated herein by reference.

### BACKGROUND OF THE INVENTION

### Field of the Invention

The present inventions relate to methods and systems that provide for the development and utilization of smaller scale chemical synthesis systems. In particular, the present inventions relate to new and improved methods, devices and systems for recovering and converting waste gases, such as flare gas, into useful and economically viable materials.

The term “flare gas” and similar such terms should be given their broadest possible meaning, and would include gas generated, created, associated or produced by, or from, oil and gas production, hydrocarbon wells (including shale, conventional and unconventional wells), petrochemical processing, refining, landfills, waste water treatment, livestock production, and other municipal, chemical and industrial processes. Thus, for example, flare gas would include stranded gas, associated gas, landfill gas, vented gas, biogas, digester gas, small-pocket gas, and remote gas.

Typically, the composition of flare gas is a mixture of different gases. The composition can depend upon the source of the flare gas. For instance, gases released during oil-gas production mainly contain natural gas. Natural gas is more than 90% methane (CH₄) with ethane and smaller amounts of other hydrocarbons, water, N₂ and CO₂ may also be present. Flare gas from refineries and other chemical or manufacturing operations typically can be a mixture of hydrocarbons and in some cases H₂. Landfill gas, biogas or digester gas typically can be a mixture of CH₄ and CO₂, as well as small amounts of other inert gases. In general, flare gas can contain one or more of the following gases: methane, ethane, propane, n-butane, isobutane, n-pentane, isopentane, n-hexane, ethylene, propylene, 1-butene, carbon monoxide, carbon dioxide, hydrogen sulfide, hydrogen, oxygen, nitrogen, and water.

The majority of flare gas is produced from smaller, individual point sources, such as a number of oil or gas wells in an oil field, a landfill, or a chemical plant. Prior to the present inventions flare gas, and in particular flare gas generated from hydrocarbon producing wells, and other smaller point sources, was burned to destroy it, and in some instances may have been vented directly into the atmosphere. This flare gas could not be economically recovered and used. The burning or venting of fare gas, both from hydrocarbon production and other endeavors, raises serious concerns about pollution and the production greenhouse gases.

As used herein unless specified otherwise, the terms “syngas” and “synthesis gas” and similar such terms should be given their broadest possible meaning and would include gases having as their primary components a mixture of H₂ and CO; and may also contain CO₂, N₂, and water, as well as, small amounts of other materials.

As used herein unless specified otherwise, the term “product gas” and similar such terms should be given their broadest possible meaning and would include gases having H₂, CO and other hydrocarbons, and typically significant amounts of other hydrocarbons, such as methane.

As used herein unless specified otherwise, the term “reprocessed gas” includes “syngas”, “synthesis gas” and “product gas”.

As used herein unless specified otherwise, the terms “partial oxidation”, “partially oxidizing” and similar such terms mean a chemical reaction where a sub-stoichiometric mixture of fuel and air (i.e., fuel rich mixture) is partially reacted (e.g., combusted) to produce a syngas. The term partial oxidation includes both thermal partial oxidation (TPOX), which typically occurs in a reformer, and catalytic partial oxidation (CPOX). The general formula for a partial oxidation reaction is

$${C}_{n} {H}_{m} + \frac{n}{2} {O}_{2} ⟶ nCO + \frac{m}{2} {H}_{2}$$

As used herein unless specified otherwise, the term “CO₂e” is used to define carbon dioxide equivalence of other, more potent greenhouse gases, to carbon dioxide (e.g., methane and nitrous oxide) on a global warming potential basis of 100 years, based on Intergovernmental Panel on Climate Change (IPCC) Fifth Assessment Report (AR5) methodology. The term “carbon intensity” is taken to mean the lifecycle CO₂e generated per unit mass of a product.

As used herein, unless specified otherwise, the terms % and mol % are used interchangeably and refer to the moles of a first component as a percentage of the moles of the total, e.g., formulation, mixture, material or product.

As used herein unless specified otherwise, the recitation of ranges of values herein is merely intended to serve as a shorthand method of referring individually to each separate value falling within the range. Unless otherwise indicated herein, each individual value within a range is incorporated into the specification as if it were individually recited herein.

Generally, the term “about” as used herein unless stated otherwise is meant to encompass the greater of a variance or range of ±10% or the experimental or instrument error associated with obtaining the stated value.

As used herein, unless specified otherwise, the terms “cost,” “costs,” “price,” “prices,” “capital cost”, in general mean the amount of money that a customer is required to pay for the transfer of title or possession of a material or goods from the holder of the material or goods to the customer. Thus, cost is the expenditure required to create and sell products and services, or to acquire assets. Capital cost as used herein, is the cost of the property, equipment and facilities that makes up a plant or facility, such as a chemical processing plant. These terms should be given their definitions as used in the US Generally Accepted Principals of Accounting (GAAP) and as used in the International Financial Reporting Standards (IFRS), the entire disclosures of each of which are incorporated herein by reference.

As used herein, unless stated otherwise, the terms and symbols “dollars,” “dollar” and “$” refer to United States (US) dollars.

As used herein, unless stated otherwise, room temperature is 25° C., and standard temperature and pressure is 15° C. and 1 atmosphere (1.01325 bar). Unless expressly stated otherwise all tests, test results, physical properties, and values that are temperature dependent, pressure dependent, or both, are provided at standard temperature and pressure.

This Background of the Invention section is intended to introduce various aspects of the art, which may be associated with embodiments of the present inventions. Thus, the forgoing discussion in this section provides a framework for better understanding the present inventions, and is not to be viewed as an admission of prior art.

### SUMMARY

There is a long standing and ever increasing problem with the direct scaling of chemical processes from large to small-scale resulting in excessively large, complex, and costly systems leading to high costs for obtaining the process product. A related problem at small-scale is the requirement that “packets” of process gas are exposed to the same temperature, pressure and residence time as they flow through the process. Failure to achieve such process uniformity results in low process yield and undesired side reactions, which can reduce product purity. The present inventions address and solve, among other, these problems.

Thus, there is provided new strategies which are defined for process intensification (PI) that result in lower part count, merging functional, modular subsystems for ease of assembly and maintenance to obtain the overall simplification needed to achieve competitive product cost at these small scales. Furthermore, new PI strategies are also defined for debottlenecking limitations by removing mass/heat transfer limitations and leveraging non-thermal driving forces for separation.

Further, there is provided methods and systems where design contradictions are identified and resolved by leveraging new ideas and technologies such as 3D printing (additive manufacturing). For example, standard manufacturing techniques of packed bed reactors and tube and shell heat exchangers results in unwanted gradients at small-scale due to high surface to volume ratio. New additive manufacturing techniques, where additional complexity is available at low incremental cost, allow design of micro-channel reactors in close contact with cooling passages that adopt spiral or curved configurations in order to move heat and reactions from the center to outer part of a reactor to the inner part (and back) to achieve more average uniform Pressure-Temperature-time (P-T-t) history. Similarly, functions like reaction and separation can be combined to improve one or more of driving forces, remove bottlenecks, avoid equilibrium limitations, and reduce unit operations count, among other advantages.

Yet further, there is provided a system and method where reactive separations are used to reduce the capital cost and operating costs of downstream conversion of syngas produced in an engine-reformer to liquid products such as methanol or ammonia. The separations can be carried out in situ in the reactor or in a close-coupled recycle loop that does not require energy intensive processes such as heating, cooling or recompression. The separation process could consist of adsorption, absorption, membrane separation (using ceramic or high-temperature polymeric membranes), distillation, or the like. The reactive separation in this embodiment is used to increase the single-pass conversion and thus reduce or eliminate the need for a recycle loop, which contributes substantially to capital and operating costs.

Additionally, there is provided a system and method where product purity can be further improved by non-thermal, nonequilibrium separation of organic molecules (e.g., alcohols, ketones) from aqueous solutions, such as sonoseparation (i.e., separation processes and equipment, including chemical separations, using sound and, in particular ultrasound), which avoids the more traditional, and more energy demanding methods of separation, such as distillation. Generally, in sonoseparation, fine droplets, generated when water-alcohol mixtures are subjected to ultrasound-induced capillary waves, can be performed at ambient pressures and temperatures and are enriched in alcohol concentrations that exceed those in the bulk solution and those prescribed by vapor-liquid phase equilibria. Such processes may improve the production grade of the product stream without the costly requirements of installing and operating distillation columns (e.g., to purify crude methanol).

Moreover, there is provided a system and method where structured materials are used to reduce the capital cost and operating costs of downstream conversion of syngas produced in an engine-reformer to liquid products such as methanol or ammonia. The structured materials have the effect of increasing thermal homogeneity in a reacting mixture and increasing heat transfer rates from the reacting mixture to the cooling medium. The structure materials can be micro-channel reactors, milli-channel reactors, structured catalyst (e.g., monoliths), high-thermal-conductivity catalyst packings, fractal devices and combinations and variations of these, among others devices. Micro- and milli-reactors can have small feature sizes and complex internal geometries (e.g., fractal devices) that lend themselves well to new additive manufacturing techniques (e.g., metal 3D printing). In a related embodiment, high thermal conductivity catalyst packings are used to replace traditional catalyst beds (e.g., catalyst deposited on alumina pellets). The high temperature packing material can be a metal mesh or the like that supports or sequesters the catalyst. The catalyst can be a powder that is physically sequestered in the fine metal mesh packing or deposited using one or more catalyst deposition techniques (e.g., wash coating, wet impregnation, etc.).

Thus, there is provided a small-scale, low capital intensity (CI) plant for converting a syngas into a higher-value product, the plant having: a reactor unit configured to receive a flow of a syngas; wherein the reactor unit is configured to convert the syngas into a liquid product; wherein the reactor unit is a small-scale processing unit; and, wherein the reactor unit has a low CI. In addition, this plant may also have an air inlet for receiving a flow of air; a flare gas inlet for receiving a flow of a flare gas; a reformer in fluid communication with the air inlet and flare gas inlet; wherein the reformer is configured to receive the flows of the flare gas and air; and, the reformer configured to convert the air and flare gas into the syngas, and thereby provide the flow of the syngas to the reactor.

Moreover, there is provided these plants, systems and processes having one or more of the following features: wherein the reactor unit is a two-stage unit; wherein the reactor unit has a means for reactive separation; wherein the reactor unit has a means for reactive separation, wherein the means for reactive separation has one or more of a reactive adsorption device, a reactive distillation device, and a reactive membrane device; wherein the reactor unit has a micro-channel reactor; wherein the reactor unit has a milli-channel reactor; wherein the reactor unit has a structured catalyst; wherein the reactor unit has a high-thermal-conductivity catalyst packing; wherein the reactor unit has a, fractal device; wherein the CI is less than about $110,000/bpd; wherein the CI is from about $110,000/bpd to $45,000/bpd; wherein the capacity is less than about 1,000 bpd; wherein the capacity is from about 2 bpd to 900 bpd; where in the liquid product has methanol; where in the liquid product consists of refined grade methanol; wherein the liquid product has ammonia; and, wherein the liquid product consists essentially of ammonia.

Additionally, there is provided a small-scale, low capital intensity (CI) plant for converting a flare gas into methanol, the plant having: an air inlet for receiving a flow of air; a flare gas inlet for receiving a flow of a flare gas; a reformer in fluid communication with the air inlet and the flare gas inlet; wherein the reformer is configured to receive the flows of the flare gas and air; the reformer in fluid communication with a reactor unit; wherein the reactor unit is configured to receive a flow of the syngas from the reformer; and wherein the reactor unit is configured to convert the syngas into methanol; the reactor unit having a reactive separation process; wherein the reactor unit is a small-scale processing unit.

Further, there is provided these plants, systems and processes having one or more of the following features: wherein the reactive separation process has a sweep; wherein the sweep has a liquid sweep; wherein the sweep has a gaseous sweep; wherein the reactive separation process has a reactive adsorption; wherein the reactive separation process has a reactive distillation; wherein the reactive separation process has a reactive membrane separation; and, having a methanol refining unit.

In addition, there is provided a small-scale, low capital intensity (CI) plant for converting a flare gas into methanol, the plant having: an air inlet for receiving a flow of air; a flare gas inlet for receiving a flow of a flare gas; a reformer in fluid communication with the air inlet and the flare gas inlet; wherein the reformer is configured to receive the flows of the flare gas and air; the reformer in fluid communication with a reactor unit; wherein the reactor unit is configured to receive a flow of the syngas from the reformer; and wherein the reactor unit is configured to convert the syngas into methanol; and, the reactor unit having a sonoseparator; wherein the reactor unit is a small-scale processing unit.

Moreover, there is provided a small-scale, low capital intensity (CI) plant for converting a flare gas into methanol, the plant having: an air inlet for receiving a flow of air; a flare gas inlet for receiving a flow of a flare gas; a reformer in fluid communication with the air inlet and the flare gas inlet; wherein the reformer is configured to receive the flows of the flare gas and air; the reformer in fluid communication with a reactor unit; wherein the reactor unit is configured to receive a flow of the syngas from the reformer; and wherein the reactor unit is configured to convert the syngas into methanol; and, the reactor unit having a microchannel reactor; wherein the reactor unit is a small-scale processing unit.

Further, there is provided these plants, systems and processes having one or more of the following features: having a microchannel reactor; wherein the microchannel reactor has a plurality of cooling plates and a plurality of reaction plates; and, wherein the microchannel reactor has a reaction plate with sweep.

Still further, there is provided a small-scale, low capital intensity (CI) plant for converting a flare gas into methanol, the plant having: an air inlet for receiving a flow of air; a flare gas inlet for receiving a flow of a flare gas; a reformer in fluid communication with the air inlet and the flare gas inlet; wherein the reformer is configured to receive the flows of the flare gas and air; the reformer in fluid communication with a reactor unit; wherein the reactor unit is configured to receive a flow of the syngas from the reformer; and wherein the reactor unit is configured to convert the syngas into methanol; and, the reactor unit having a high thermal conductivity (HTC) catalyst bed; wherein the reactor unit is a small-scale processing unit.

Yet additionally, there is provided these plants, systems and processes having one or more of the following features: having an HTC bed; wherein the HTC bed has catalyst aggregates loaded into a metal foam support; wherein the catalyst support is aluminum; wherein the relative density of the foam is less than 10%; wherein the methanol is refined grade methanol; wherein the CI is less than about $110,000/bpd; wherein the CI is from about $45,000/bpd to $110,000/bpd; wherein the capacity is less than about 1,000 bpd; wherein the capacity is from about 2 bpd to 900 bdp; wherein the plant is an onsite plant and located adjacent to a source of flare gas; wherein the source of flare gas is an oil well; and, wherein the reactor unit has a catalytic bed reactor having a high thermal conductivity support.

Moreover, there is provided a method of onsite conversion of a flare gas to a liquid product using a small-scale, low capital intensity (CI) plant, the method having: receiving a flow of a flare gas from a flare gas source; providing the flare gas flow to a reformer engine; converting the flare gas flow in the reformer engine into a syngas, thereby providing a syngas flow; providing the syngas flow to a reactor unit; processing the syngas into a liquid product in the reactor unit, wherein the processing has a reactive separation process.

Yet additionally, there is provided these plants, systems and processes having one or more of the following features: wherein the reactor unit has a first reactor and a second reactor, and the processing is a two-stage process using the first reactor and the second reactor, and wherein the reactive separation process takes place in the second reactor; wherein the reactive separation process has a reactive adsorption; wherein the reactive separation process has a reactive distillation; wherein the reactive separation process has a reactive membrane separation; wherein the processing has using a catalytic bed reactor having a high thermal conductivity support; wherein the processing has using a microchannel reactor; further having a sonoseperation process to purify the liquid product; wherein the source of the flare gas is an oil field and the method is carried out at the oil field; wherein the CI is less than about $110,000/bpd; wherein the CI is from about $45,000/bpd to $110,000/bpd; wherein less than about 1,000 bpd of liquid product is produced; wherein from about 2 bpd to 900 bdp of liquid product is produced; where in the liquid product has methanol; where in the liquid product consists of refined grade methanol; wherein the liquid product has ammonia; wherein the liquid product consists essentially of ammonia.

Moreover, there is provided a method of designing a small-scale, low capital intensity (CI) plant for converting a flare gas into methanol, the method including: selecting components of a reactor unit to conduct a syngas to methanol process; wherein the components of the reactor unit include components to conduct a reactive separation process; optimizing the syngas to methanol process, the reactive separation process, or both, to provide a design for reactor unit having a small-scale and a low CI.

Yet additionally, there is provided these methods of designing having one or more of the following features: wherein the reactor unit has a CI of less than about $110,000/bpd; wherein the reactor unit has a CI from about $45,000/bpd to $110,000/bpd; wherein the reactor unit has a capacity of less than about 1,000 bpd; wherein the reactor unit has a capacity from about 2 bpd to 900 bdp; and, wherein the plant is configured to provide a refined grade methanol.

### BRIEF DESCRIPTION OF THE DRAWINGS

FIG. 1 is a chart showing an embodiment of the variation in capital intensity of gas-to-liquids (GTL) plants with plant scale in accordance with the present inventions.

FIG. 2 is a schematic diagram of an embodiment of a system and method showing basic gas-to-methanol process configuration with no process intensification.

FIG. 3 is a schematic diagram of an embodiment of a system and method showing gas-to-methanol process configuration with reactive separation of products in accordance with the present inventions.

FIG. 4 is a schematic diagram of an embodiment of a system and method showing gas-to-methanol process configuration with reactive separation of byproducts in accordance with the present inventions.

FIG. 5 a schematic diagram of an embodiment of a system and method showing gas-to-methanol process configuration with sonoseparation of crude methanol to refined methanol in accordance with the present inventions.

FIG. 6 is a schematic prospective view of an embodiment of a microchannel reactor showing cooling plates and two options for reaction plates (with and without a sweep stream for reactive separation) in accordance with the present inventions.

FIG. 7 is a schematic diagram of a multi-tubular boiling water reactor with a high-thermal-conductivity media in the tubes containing the catalyst in accordance with the present inventions.

FIG. 8 is a graph of the temperature profiles within and upstream of a methanol synthesis tubular reactor bed, composed of catalysts aggregates either freely loaded or packed in a high-thermal-conductivity support

### DESCRIPTION OF THE PREFERRED EMBODIMENTS

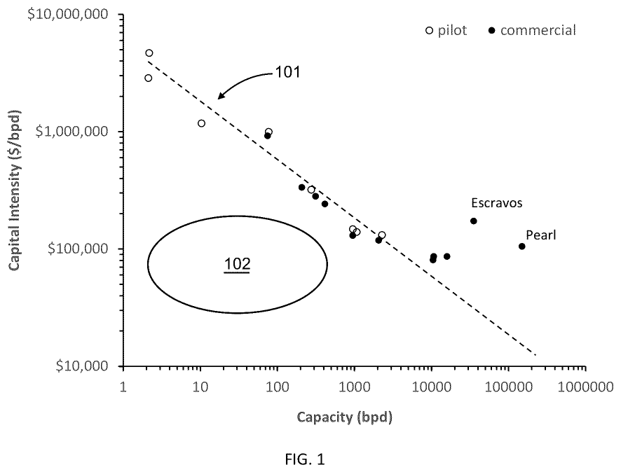

In general, embodiments of the present inventions relate to new systems and devices for chemical synthesis that reduce the capital intensity (e.g., capital cost per unit of throughput in, for example, units of Untied States Dollars (USD) per barrels per day (bpd) of capacity) of smaller chemical production facilities. The cost of capital equipment tends to scale sub-linearly with capacity at approximately six-tenths power. Thus, traditionally, and prior to the present inventions, larger processing equipment (e.g., bigger reactors, bigger chambers, bigger separators, etc.) had much better capital intensity (i.e., lower values) than smaller processing equipment. Thus, traditionally, and prior to the present inventions, the result of this scaling is that capital intensity is higher for smaller pieces of equipment than for larger pieces of the same type of equipment. This traditional scaling paradigm can be seen, for example, in scaling of gas-to-liquids (GTL) processes as shown in dashed line 101 of FIG. 1.

In general capital intensity (CI), is the ratio of the cost of a particular piece of equipment, or the cost of all of the pieces of equipment needed to conduct a particular manufacturing process, e.g., a chemical process, vs. the production or output capacity of that piece of equipment or pieces of equipment. In particular, CI as used herein, unless expressly stated otherwise, is the cost in US dollars for a particle piece of equipment or particular pieces of equipment (i.e., a production unit), vs the capacity or output of a product (e.g., rated capacity, projected capacity or actual operating capacity) in bpd from that particular piece of equipment or production unit. As such, the units for CI are US dollars/bpd.

Embodiments of the present inventions provide the ability to greatly reduce the CI for smaller pieces of equipment (e.g., smaller capacity) and smaller production units (e.g., smaller capacity). This, among other things, provides for much greater cost effectiveness, for the use and placement of small onsite equipment and processing units. For example, by having a small processing unit, having a low CI, at a remotely located low value source material supply, the low value source material can be cost effectively processed into a high value material, greatly reducing shipping costs from the remote location.

Thus, by way of example, embodiments of the present invention provide the ability to reduce the CI of small-scale equipment and production units by 50% or more, 60% or more, 70% or more, and 90% or more, and be from 30% to 90%, from 40% to 80%, from 60% to 80%, and from 50% to 90%. (As used herein a percentage reduction is the amount of decrees divided by the original value times 100. Thus a 70% decrease for a CI of 150 $/bpd would result in a CI of 45 $/bpd.) Further, these small-scale equipment and production units have a capacity of 2 bpd to 5,000 bpd, 5 bpd to 1,000 bpd, from 5 bpd to 700 bpd, from 10 bpd to 200 bpd, less than 1,000 bpd, less than 700 bpd, less than 500 bpd, less than 100, bpd, and less than 10 bpd.

Similarly, these small-scale equipment and processing units can have Cis that are the same as the Cis for larger scale (i.e., higher capacity) versions of such equipment and processing units. For example, a 1,000 bpd processing unit can have the same CI as a 100,000 bpd processing unit. Thus, embodiments of the present inventions can have the same CI as pieces of equipment and processing units that have capacities that are from 2× to 1,000× larger, from 2× to 10× larger, from 10× to 100× larger, from 5× to 50× larger, at least 2× larger, at least 10× larger, and at least 100× larger.

As used herein “small-scale” equipment and processing units refers to capacities that are smaller than 5,000 bpd, smaller than 2,000 bpd, smaller than 1,000 bpd, smaller than 500 bpd, smaller than 100 bpd, smaller than 50 bpd, smaller than 10, bdp, and smaller than 5 bpd, and from 2 bpd to 5,000 bpd, from 2 bpd to 1,000 bpd, from 5 bpd to 700 bpd, from 10 bpd to 200 bpd. As used herein “low CI” refers to Cis that are less than 250,000 $/bpd, less than 200,000 $/bpd, less than 100,000 $/bpd, less than 50,000 $/bpd, and from 40,000 $/bpd to 200,000 $/bpd, from 50,000 $/bpd to 150,000 $/bpd, and from 50,000 $/bpd to 110,000 $/bpd. Thus, embodiments of the present inventions include embodiments of these small-scale equipment and processing units having these low Cis.

As used herein, when comparing CI or capacity, the comparison is done for the same type of equipment or processing unit and for the same product that is being provided or produced by that equipment or processing unit.

Thus, embodiments of the present inventions break, or overcome, the long standing scaling paradigm, by providing equipment and processing units that have both small-scale and low CI.

Generally, embodiments of the present invention process intensification (PI), as set forth in this specification to reduce capital costs for small-scale equipment and production units. Thus, and in general, an embodiment of an approach to reduce the capital intensity is to leverage the principles of PI to reduce the capital costs for chemical production, shifting off the traditional line 101 in FIG. 1 and into the region shown approximately by the oval 102. In general, in addition to improving CI for small scale equipment and production units, embodiments off the present inventions can provide improved operating costs and improved efficiencies. Other potential advantages of these embodiments can include improving process safety, thereby reducing energy intensity and minimizing carbon footprint.

Although, present specification generally addresses improvements to systems, devices and methods to recover in an economical fashion usable fuels from flare gas, and in particular, in an embodiment, to achieve such recovery at smaller, isolated or remote locations or point sources for the flare gas, it is understood that embodiments of the present inventions find application and benefits in any industrial (e.g., chemical, hydrocarbon, waste management, etc.) or agricultural (e.g., livestock, food processing, etc.) processes setting, and in particular, where onsite small-scale systems are advantageous.

In general, preferred embodiments of the present inventions take uneconomic hydrocarbon-based fuels at a wellhead and remote locations that are primarily gaseous hydrocarbons and convert them to a more valuable, easily condensable or liquid compounds, such as methanol. One source of source fuel (e.g., waste gases) could be associated gas or flare gas, which is produced as a byproduct at oil wells. Another source could be biogas from landfill or anaerobic digesters.

Generally, embodiments of the present inventions are directed to one or more of three methods and device providing process intensification that can be used together or separately, or with other methods and devices: (1) Maximizing synergistic effects from partial processes, such as, Functional PI for Reduction of Costs; (2) Optimize the driving forces at every scale, in particular by harnessing non-thermal driving forces, such as Thermodynamic PI for Reduction of Costs; and (3) Giving each molecule the same processing experience, such as Spatial PI for Reduction of Costs.

Functional PI for Reduction of Costs

The first method and devices relate to the functional domain of PI and generally seeks to integrate process steps in new or different ways to leverage synergies, increase product yield, increase energy efficiency, reduce the physical footprint of a process, and combinations and variations of these. Examples include heat-exchanger (HEX) reactors, hybrid distillation, and especially reactive separations. In the context of small, distributed production of liquid chemicals (e.g., small onsite equipment and production units) such as methanol or ammonia, reactive separations can be used to combine the reaction and separation steps to overcome the equilibrium limitation and increase the single-pass yield. In commercial methanol and ammonia processes, which are both equilibrium-limited and involve a net decrease in moles upon reaction, the reactor pressure is increased to reach acceptable single-pass conversions, however, this approach leads to increased capital cost for higher pressure equipment and increased operating costs associated with compressing gas phase reactant streams. Because the single-pass conversion is still low for these processes, an energy-intensive downstream separation and recycle loop is used to isolate the product and return unreacted reactants back to the reactor inlet. Large recycle loops are costly both in terms of capital costs (e.g., reactor vessels must be sized larger to accommodate the increased inlet mass flows due to recycle) and operating costs (e.g., ammonia and methanol are separated first by cooling and condensing the reactor effluent and then the recycle stream is reheated before returning to the reactor).

In embodiments, methods and devices to selectively remove the product from the reactor in situ or in a close coupled fashion are used. This approach is generally referred to as reactive separations. Removing the products from an equilibrium limited reaction has the net effect of inducing additional conversion of reactants according to Chatelier's principle. The separation can be done using various separation technologies such as adsorption, absorption, distillation, and membranes yielding hybrid processes called reactive adsorption, reactive distillation, and membrane reactors. Reactive separations can be used to increase conversion at a fixed pressure or to achieve the same conversion as a traditional reactor at lower pressure.

Thermodynamic PI for Reduction of Costs

The second method and devices relate to the thermodynamic domain of PI and generally seeks to optimize driving forces. In particular for embodiments of the present inventions, the principle applies to switching from thermal, equilibrium separations of the products to a non-thermal, nonequilibrium alternative. As an example, separations in chemical manufacturing typically constitute the majority of the equipment cost and operating costs in a chemical plant. These separation processes account for 10-15% of the world's energy consumption. Although various separation technologies are used, the chemical process industry is dominated by distillation, which is very energy intensive. In the case of distillation, the driving force is heat and the separation depends on differences in the volatility of the components within a mixture. There is an opportunity for PI by harnessing non-thermal driving forces. One attractive option for methanol-water separation (i.e., the upgrading of crude methanol to a more refined grade upon water removal) is sonoseparation. The advantages include lower energy intensity, an opportunity for process electrification, and improved packaging for small, modular deployed applications (in contrast to distillation towers, which must be vertically configured with heights dictated by the tens of theoretical stages necessary to achieve good separation).

Spatial PI for Reduction of Costs

The third methods and devices relate to the spatial domain of PI and generally seeks to give each molecule the same processing experience (e.g., well-defined and homogeneous temperature-pressure-time histories for packets of fluid passing through the process). Other advantages include inducing high heat and mass transfer rates and increasing the surface area for heat and mass transfer. Examples of process equipment for PI in the spatial domain for reactions include micro-channel reactors, milli-channel reactors, structured catalyst (e.g., monoliths), high-thermal-conductivity catalyst packings, and fractal devices. Because of the complex internal geometries of micro-channel and other devices, these approaches often lend themselves to additive manufacturing (i.e., 3D printing) because they are more difficult to produce with traditional subtractive manufacturing techniques (e.g., require increased parts count).

Methanol and ammonia synthesis are both highly exothermic, so managing the exotherm is critical to achieving high performance in the process. Thermal management of the reactor is critical as local hot-spots lead to sintering of the metal catalyst nanoparticles, which is a primary mechanism for catalyst deactivation through the consequent loss of active surface sites. Commercial methanol synthesis reactors in the prior art use boiling water reactors (BWRs) that comprise a bundle of long, narrow tubes submersed in boiling water at elevated pressure. The catalyst pellets are packed inside the tubes and the coolant water is on the outside of the tubes in a pressurized vessel. Micro-channel reactors use compact micro-channel geometries to increase the heat transfer area between the catalyst bed and the cooling water; these micro-channels also decrease the characteristic distance for heat transfer inside the tubes (i.e., the tube inner diameter), which increases the thermal homogeneity within the catalyst beds. The heat transfer rates are measured to be up to 10× higher for microchannel devices. The improved heat transfer allows for a compact and precisely controlled process that can handle the highly exothermic nature of the methanol and ammonia synthesis reactions. While these two chemistries are discussed in detail, this approach also applies to other chemistries with no loss of generality. Micro-channel devices are also well suited for high pressure applications because the hoop stress (and thus the required wall thickness) scales linearly with channel diameter (or hydraulic diameter). The net effect means small channels require thinner tube walls than larger tubes or channels, which leads to a reduction in material costs. These reductions in material costs can be substantial considering that high-performance materials (e.g., stainless steel), which cost more than more conventional materials (e.g., carbon steel), are often required for many types of reactor equipment. In summary, micro-channel devices increase heat transfer rates and heat transfer area compared to conventional geometries such as BWRs.

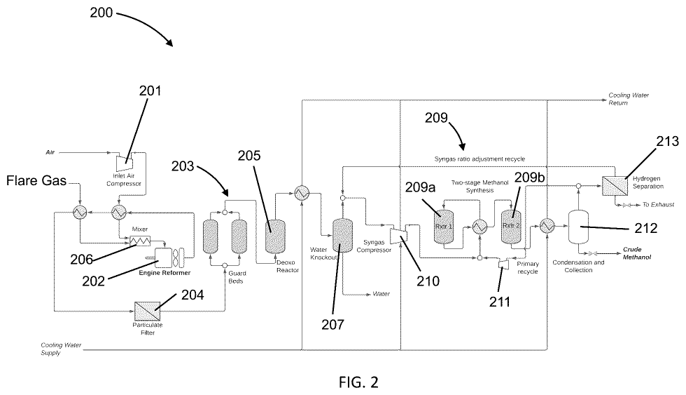

Turning to FIG. 2, there is shown a schematic production flow diagram of a gas-to-liquids process and system that converts flare gas into a syngas intermediate and then to a methanol product via high pressure, catalytic reaction. Optionally, the synthesis unit is a two-stage unit 209 with a first reactor unit 209a and a second reactor unit 209b.

The system 200 has an inlet air compressor 201, a mixer 206 for mixing the incoming flow of air and the incoming flow of flare gas. The system 200 has an engine reformer 202 for converting the flare gas-air mixture into a syngas. The system 200 also has a particulate filter 204, guard beds 203, deoxo reactor 205. The portion of the system, directed to converting the syngas to methanol (e.g., the synthesis unit) has a water knockout 207, a compressor 210, a two-stage reactor unit 209, having a first reactor 209a and a second reactor 209, a compressor 211, a hydrogen separation unit 213 and a condensing unit 212.

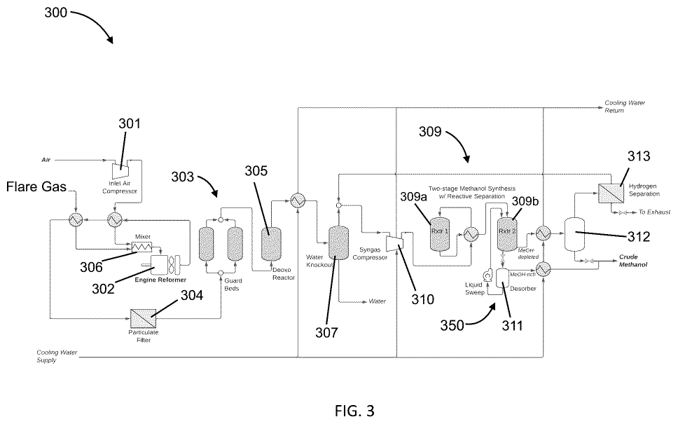

Generally, for configurations of the type shown in the embodiment of FIG. 2, product streams are expanded through valves (or backpressure regulators) in a Joule-Thompson (i.e., isenthalpic) process prior to product condensation and collection of crude methanol. Low single-pass conversions have a recycle loop for unreacted molecules and consequent compression of this gas phase effluent stream back to the inlet of the reactor, which must be sized to accommodate this additional recycle volume. This recycle loop thus leads to costs associated with installing and operating a compressor and with increased sizing of the reactor vessel. Instead, such a process step can be eliminated through the reactive separation of products, either through the removal the primary, desired product (e.g., methanol), the removal of undesired byproducts (e.g., water) or both. Embodiments of systems using reactive separation methodologies are shown, for example, in the embodiments of the type of systems and processes shown in FIGS. 3, 4, and 5.

Turning to FIG. 3, there is shown a schematic production flow diagram of a gas-to-liquids process and system that converts flare gas into a syngas intermediate and then to a methanol product via high pressure, catalytic reaction. The synthesis unit is a two-stage unit 309, with a first reactor unit 309a and a second reactor unit 309b and a reactive separation loop 350.

The system 300 has an inlet air compressor 301, a mixer 306 for mixing the incoming flow of air and the incoming flow of flare gas. The system 300 has an engine reformer 302 for converting the flare gas-air mixture into a syngas. The system 300 also has a particulate filter 304, guard beds 303, deoxo reactor 305. The portion of the system, directed to converting the syngas to methanol (e.g., the synthesis unit) has a water knockout 307, a compressor 310, a two-stage reactor unit 309, having a first reactor 309a and a second reactor 309, a compressor 311, a hydrogen separation unit 313 and a condensing unit 312. The system 300 has a reactive separation loop 350 (e.g., a methanol separation loop) that has desorber 311.

The process uses a two-stage methanol synthesis reactor 309 with reactive separation in the second stage (Rxtr 2) 309b only. The first stage (Rxtr 1) 309a generally does not approach equilibrium and thus does not warrant reactive separation. The embodiment shown in this FIG. 3 is reactive absorption or membrane separation with a liquid sweep. The reactor 309b could be a trickle bed or a membrane reactor with the liquid absorbent (sweep) on the permeate side of the membrane. Methanol is selectively removed from the reactor in situ resulting in a methanol-depleted gaseous stream consisting primarily of unreacted syngas and a methanol-rich absorbent stream. The depletion of methanol in the gaseous stream leads to increased methanol yield as reaction equilibrium limitations are removed. As a consequence, the primary recycle loop (e.g. FIG. 2) is no longer required because of the improved single-pass conversion, eliminating the associated costs for installing and operating a compressor 211. The methanol-rich absorbent stream is depressurized as it passes through a valve, after which, methanol desorbs and then condenses in subsequent steps in the desorber 311. The absorbent, now in a regenerated state, is pumped back to reaction pressure and recirculated to the reactor inlet. Work associated with pumping the liquid absorbent is minimal, compared to that required for syngas compression, because the absorbent is nearly incompressible thus lowering operating costs; similarly, capital costs associated with installing a liquid pump (for absorbent handling) are lower than those for a syngas compressor. Any methanol that does not partition into the absorbent is condensed out of the gas phase in a downstream separation step and combined with the methanol product stream.

Thus, embodiments of the type shown in FIG. 3, and in particular the syntheses unit 309 with the reactive separation loop 350, can have a low CI, can be a small-scale processing unit, and can be both small-scale and have a low CI. Embodiments of the type shown in FIG. 3, and in particular the syntheses unit 309 with the reactive separation loop 350 can have the percentage reductions in CI ass set forth above in this Specification. Similarly, these small-scale equipment and processing units, of the type shown in the embodiment of FIG. 3 can have Cis that are the same as the Cis for larger scale (i.e., higher capacity) versions of such equipment and processing units as set forth above in this Specification.

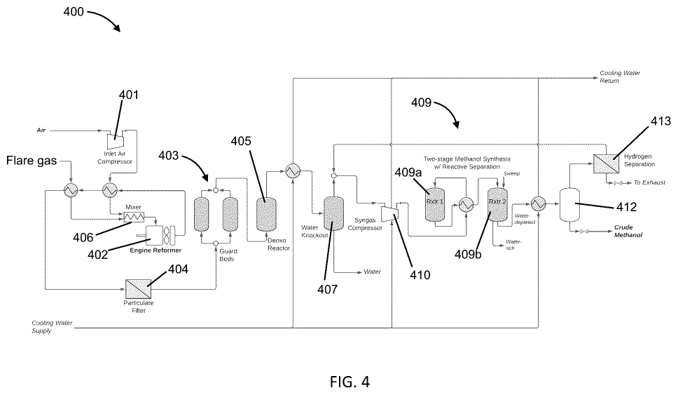

Turning to FIG. 4, there is shown a schematic production flow diagram of a gas-to-liquids process and system that converts flare gas into a syngas intermediate and then to a methanol product via high pressure, catalytic reaction. The synthesis unit is a two-stage unit 409, with a first reactor unit 409a and a second reactor unit 409b and a reactive separation of byproducts.

The system 400 has an inlet air compressor 401, a mixer 406 for mixing the incoming flow of air and the incoming flow of flare gas. The system 400 has an engine reformer 402 for converting the flare gas-air mixture into a syngas. The system 400 also has a particulate filter 404, guard beds 403, deoxo reactor 405. The portion of the system, directed to converting the syngas to methanol (e.g., the synthesis unit) has a water knockout 407, a compressor 410, a two-stage reactor unit 409, having a first reactor 409a and a second reactor 409, a hydrogen separation unit 413 and a condensing unit 412.

The process uses a two-stage methanol synthesis reactor 409 with reactive separation in the second stage (Rxtr 2) 409b only. The first stage (Rxtr 1) 409a generally does not approach equilibrium and thus does not warrant reactive separation. The embodiment shown in this FIG. 4 is membrane separation with a gaseous sweep. The membrane reactor could use a polymeric or ceramic membrane material that is perm-selective to water and a sweep gas (e.g., air) on the permeate side of the membrane. Water, which is a byproduct of the CO₂ hydrogenation reaction to methanol, is selectively removed from the reactor in situ resulting in a water-depleted gaseous stream consisting primarily of unreacted syngas and a water-rich sweep gas. The depletion of water in the gaseous stream leads to increased methanol yield (from CO₂ hydrogenation) as reaction equilibrium limitations are removed. Lower water partial pressures can also remove equilibrium limitations present in reverse water-gas shift reactions, leading to increased yield of CO, a more reactive reactant molecule in methanol formation (via CO hydrogenation). As such, this approach is particularly attractive for CO₂-rich syngas streams, such as those produced from partial oxidation. Taken together, a primary recycle loop as seen in the embodiment of FIG. 2 is no longer required because of the improved single-pass conversion. In comparison to embodiments, such as the type shown in FIG. 3, regeneration of the sweep stream (e.g., air in the embodiment of FIG. 4) is not required, avoiding the costs associated with absorbent handling.

Thus, embodiments of the type shown in FIG. 4, and in particular the syntheses unit 409 with the reactive separation sweep, can have a low CI, can be a small-scale processing unit, and can be both small-scale and have a low CI. Embodiments of the type shown in FIG. 4, and in particular the syntheses unit 409 with the reactive separation sweep can have the percentage reductions in CI ass set forth above in this Specification. Similarly, these small-scale equipment and processing units, of the type shown in the embodiment of FIG. 4 can have CIs that are the same as the Cis for larger scale (i.e., higher capacity) versions of such equipment and processing units as set forth above in this Specification.

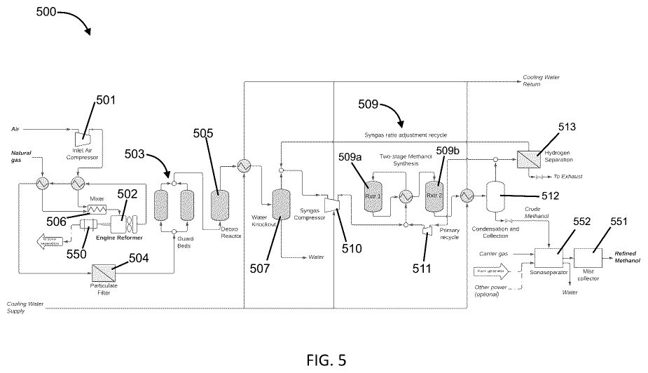

Turning to FIG. 5, there is shown a schematic production flow diagram of a gas-to-liquids process and system that converts flare gas into a syngas intermediate and then to a methanol product via high pressure, catalytic reaction. The synthesis unit is a two-stage unit 509, with a first reactor unit 509a and a second reactor unit 509b and the sonoseparation of the condensate stream, consisting of crude methanol.

The system 500 has an inlet air compressor 501, a mixer 506 for mixing the incoming flow of air and the incoming flow of flare gas. The system 500 has an engine reformer 502 for converting the flare gas-air mixture into a syngas. The system 500 also has a particulate filter 504, guard beds 503, deoxo reactor 505. The portion of the system, directed to converting the syngas to methanol (e.g., the synthesis unit) has a water knockout 507, a compressor 510, a two-stage reactor unit 509, having a first reactor 509a and a second reactor 509, a compressor 511, a hydrogen separation unit 513 and a condensing unit 512. The system 500 also a system to refine the methanol produced from the synthesis unit (e.g. methanol refining unit). The methanol refining unit has a sonoseparator 552, a mist collector 551 and a generator 550.

The sonoseparation step proceeds by exposing this liquid stream, which can contain up to 10 vol % water, to ultrasound waves, in the sonoseparator 552, generating a mist composed of micron-sized droplets, enriched in methanol, that are entrained in inert gas flow (e.g., nitrogen). The embodiment shown in this FIG. 5 includes a collection set-up (e.g., mist collector 551) that facilitates the nucleation of a liquid phase from the flowing effluent mist. The recovered liquid in these mist collectors can be composed of highly pure, refined grade methanol (99.85% methanol), achieved without the need for cost-intensive installation and operation of thermal, equilibrium-limited separation processes, such as distillation columns. Power for the ultrasound actuator in the sonoseparator 552 can be supplied by a generator 550 coupled to the shaft of the upstream engine reformer 502 (as shown in FIG. 5), or by expansion of the tail gas in a turbo-expander coupled to a generator, by renewable sources (e.g., wind, solar), or by other sources. The water-rich stream exiting the sonoseparator 552 that is depleted in methanol can be used elsewhere in the process, stored, used to stimulate a well, further purified on-site, disposed on-site, transported and processed remotely, used for irrigation, or the like.

Thus, embodiments of the type shown in FIG. 5, and in particular the syntheses unit 509 with the sonoseparation system, can have a low CI, can be a small-scale processing unit, and can be both small-scale and have a low CI. Embodiments of the type shown in FIG. 5, and in particular the syntheses unit 509 with the sonoseparation system can have the percentage reductions in CI ass set forth above in this Specification. Similarly, these small-scale equipment and processing units, of the type shown in the embodiment of FIG. 5 can have CIs that are the same as the Cis for larger scale (i.e., higher capacity) versions of such equipment and processing units as set forth above in this Specification.

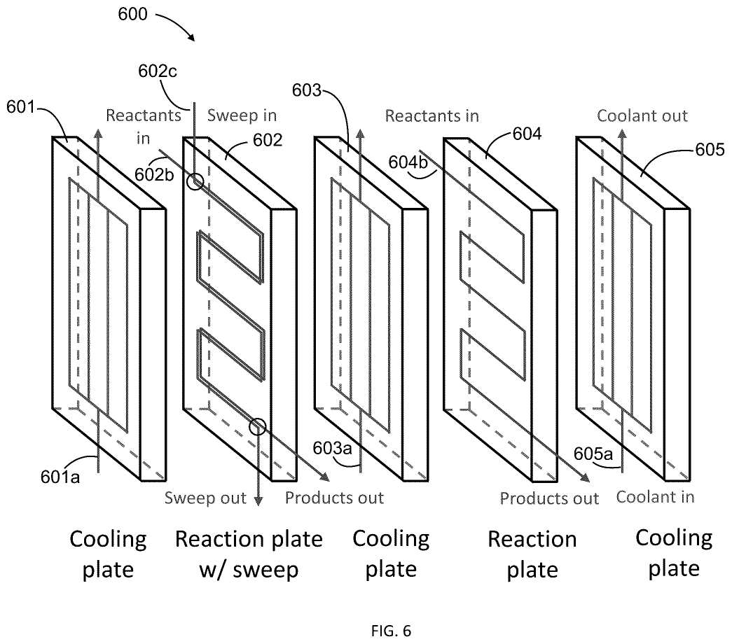

Turning to FIG. 6 there is shown an exploded perspective schematic view of an embodiment of a micro-channel reactor geometry with cooling. This micro-channel reactor can be used in any gas-to-liquid systems and processes, in particular flare gas to methanol systems and processes. Further, this micro-channel reactor can be used as the reactor in any of the embodiments of the types of systems and processes shown in FIGS. 2 to 5. Preferably, this type of micro-channel reactor would be the second reactor in the system, or could be a third or additional reactor.

The micro-channel device 600 has two reaction units or plates 602, 604 that have cooling units or plates 601, 603, 605 on either side of the reaction units. It being understood that the figure is an exploded view, and thus, in actuality the surfaces of the units are in physical and thermal contact with the adjacent units. While 5 units are shown it is understood that there could be 3 units (one reaction and two cooling), 7 units, 9 units, 11 units, etc. While it is preferred that each reaction unit has a cooling unit on both sides (as shown in FIG. 6), embodiments may have configurations where the outer most reaction unit has only a single cooling unit adjacent to it.

In the embodiment of FIG. 6, the flow of reactants is shown by arrows 602b, 604b. The flow of the reactants 602b, 604b is in a serpentine path in each of the respective reaction units 602, 604. The flow of the coolant is shown by arrows 601a, 603a, 605a. The flow of the coolants 601a, 603a, 605a in in a parallel path in each of the respective cooling units 601, 603, 605. In an exothermic reaction, like ammonia or methanol synthesis, heat is transferred from reactants to the coolant through the walls of the micro-channel device 600. Preferably, characteristic heat transfer lengths are kept short, and heat transfer area per unit of overall volume is larger than in traditional reactor designs. The advantaged heat transfer helps to ensure uniformity of the catalyst bed temperature and mitigates hot-spot formation, which is a primary mechanism for catalyst sintering and deactivation. The coolant could be a single-phase heat transfer fluid or a boiling fluid such as water. In this example, the coolant and reactant flows are countercurrent so that, in the case of an exothermic equilibrium-limited reaction, the heat (generated from reaction) from the reactor effluent stream can be transferred to the inlet coolant fluid (exhibiting the lowest temperature) at the reaction-plate outlet, encouraging higher equilibrium conversions, although other configurations (concurrent, crossflow) are envisioned.

In an embodiment a reaction unit (one, more, than one or all) can have a second reactant, promoter, quench, or sweep stream that can be introduced into the flow of the reactant in the reactor unit or plate. Thus, for example, the reaction unit 602 is a “reaction plate with sweep”. The flow of the sweep stream is shown by arrow 602c. In a preferred embodiment, the second stream, e.g., sweep stream 602c can be an absorbent that selectively removes the products or byproducts in situ in a microchannel, reactive separation configuration. This would be viewed as a reactive separation. In an embodiment a microchannel mixer can be used near the entrance of the reaction unit to create alternating slugs or bubbles of sweep fluid and reactant fluid to provide increased interfacial surface area for mass transfer. In this embodiment the microchannel mixer can be, for example, a static mixer. In an embodiment a microchannel 24 is engage can be used to disengage the sweep fluid and the reactant fluid near the exit of the reaction unit. In a preferred embodiment, the two fluids are disengaged based on wettability or capillary forces using microchannel features (e.g., an array of posts) in the flow path.

The thickness of the channel walls required for high-pressure applications is smaller for these microchannel devices compared to larger, conventional equipment designs because of the linear scaling between hoop stress (and hence wall thickness) and channel hydraulic diameter. The thinner walls reduce the amount of material required for fabrication and hence reduces the manufacturing cost.

Manufacturing cost can further be lowered by use of additive manufacturing (e.g., 3-D printing) to form the microchannel plates. Additive manufacturing also reduces the part count by allowing channels to be printed in place as a single part without the need for a parting line as would otherwise be required for casting or conventional machining. Avoiding the parting line reduces cost (fewer parts) and reduces the likelihood of leaks at the seal (e.g., o-ring, gasket, brazed connection) joining the two halves of the microchannel plate. Optionally, the additive manufacturing may be paused to allow for manual or automated loading of catalyst in the channels and later resumed to complete the channels and encasing of the catalyst. In a preferred embodiment, the 3D printer can have multiple print heads for printing both the microchannel walls and catalyst/support in the same program/operation. The 3D printer may use various types of additive manufacturing including selective laser sintering (SLS), binder jetting, fused filament fabrication (FFF), and the like. In an embodiment, the catalyst can be printed as an ink that is deposited on the channel walls or another high-surface-area textured support that may also be 3D printed.

In the lamellar, plate architecture, of the type shown in FIG. 6, the throughput of the device can be increased by adding additional plates. Preferrably, each reaction plate can include a serpentine path to optimize residence time for the chemistry to proceed to the desired conversion. The cooling plates can preferably be configured to distribute the coolant among a collection of parallel paths to provide uniform cooling to the reaction plate. In embodiments a coolant header that is located within the reaction plate, as shown in FIG. 6 can be used to distribute the flow of the incoming coolant between the parallel channels within the plate, and then a return or collection header can also be used to combine the parallel channels into a single exist channel for the coolant to then leave the plate. The coolant headers, and in particular the distribution header, can be designed to minimize flow distribution between the parallel channels.

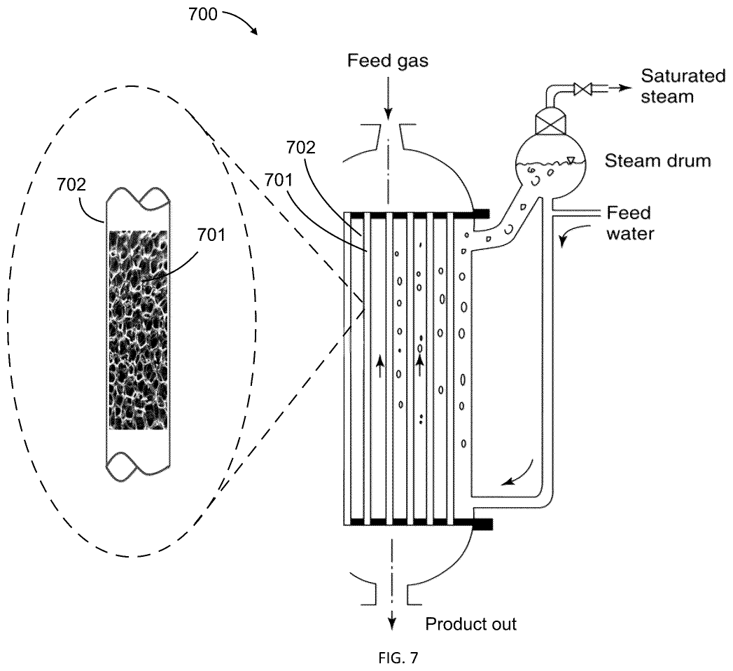

Turning to FIG. 7 there is shown a schematic diagram, with callout, showing the use of a high-thermal-conductivity catalyst support such as a fine, microfibrous nonwoven mat 701, embedded within reactor tubes 702 in a boiling water reactor (tube/shell configuration). The thermal conductivity of the support (e.g., copper, nickel, or other metals or alloys) is considerably higher than a conventional packed bed containing catalyst nominally 1-5 mm pellets or extrudates that are porous alumina (or similar) supports with active catalyst on the surface. The microfibrous catalyst supports 701 improve bed temperature uniformity, allow the use of fine catalyst particles thereby eliminating intra-pellet mass transfer limitations, and improve bed-to-wall heat transfer. In a preferred embodiment, the microfibrous supports are used with a sweep stream that selectively removes the products or byproducts in a reactive separation configuration that also incorporates microfibrous structured material internal packing. This embodiment contains elements of both PI in the functional and spatial domains. The sweep stream could be a liquid absorbent or the like.

Turning to FIG. 1, there is shown a graph plotting how capital intensity varies with process scale for prior art gas-to-liquids (GTL) plants. As shown, by line 101, capital intensity is highest for small-scale processes and follows a power-law scaling. The Pearl GTL plant (Qatar) and the Escravos GTL plant (Nigeria), two “world scale” plants, are identified on the chart, with their Cis being below $200,000/bpd. This plot demonstrates that cost reduction at small scales is needed to effectively harness smaller, stranded flare gas or associated gas resources. Thus, for example, embodiments of the present inventions provide gas-to-liquid flare gas processing systems that have Cis in the area 102, which is similar to the Cis for a world scale plant. Process intensification, either functional, thermodynamic, or spatial, can be used to reduce the capital intensity, especially at smaller scales, rather than simply scaling down the same technology used at the world scale plants.

The following examples are provided to illustrate various embodiments of the present processes and systems. These examples are provided to illustrate various embodiments of the present gas-to-liquid conversion processes and systems. These examples are for illustrative purposes, may be prophetic, and should not be viewed as, and do not otherwise limit the scope of the present inventions.

### EXAMPLES

### Example 1

A downstream synthesis reactor and process that selectively removes the product or byproduct of the synthesis reaction from unreacted synthesis gas (syngas) from the reformer to shift the equilibrium to achieve higher conversion at the same reaction conditions. The reactor and process have the following features:

The product separation occurring either in situ in the synthesis reactor or in a close-coupled fashion.

Reduction or elimination of the need for a recycle loop to the synthesis reactor, leading to reduced capital intensity via synergistic combination of process elements, thereby reducing reactor size and downstream separation complexity.

A means of separation of the products from the reactants in the reactor, which may include one of adsorption, absorption, membrane separation, distillation, or the like.

### Example 2

A downstream synthesis reactor and process that selectively removes the product or byproduct of the synthesis reaction from unreacted synthesis gas (syngas) from the reformer to shift the equilibrium to achieve higher conversion at the same reaction conditions. The reactor and process have the following features:

The product separation occurring either in situ in the synthesis reactor or in a close-coupled fashion.

Reduction or elimination of the need for a recycle loop to the synthesis reactor, leading to reduced capital intensity via synergistic combination of process elements, thereby reducing reactor size and downstream separation complexity.

A means of separation of the products from the reactants in the reactor, which may include one of adsorption, absorption, membrane separation, distillation, or the like.

A means of regenerating a sorbent or sweep stream to recover the product and return the sorbent/sweep material to the reactor.

Using outputs from the engine-reformer to provide heat, electricity, shaft power, or pneumatic/hydraulic pressure to the downstream reactive separation process.

### Example 3

A sonoseparation method and devices that upgrades crude methanol to refined methanol using a non-thermal method, as an alternative to conventional distillation.

### Example 4

A sonoseparation method and device that upgrades crude methanol to refined methanol using a non-thermal method, as an alternative to conventional distillation. The method and device have one or more of the following features:

The refined methanol meeting the purity specification (99.85%) for Grade methanol after upgrading with sonoseparation.

The electric power for the ultrasonic actuators being produced by a generator coupled to the shaft of the engine reformer.

A means of also accepting other electric power for the sonoseparation, such as renewable power produced by turbo-expander, solar, wind or other source.

### Example 5

A device and method using a structured material in the downstream synthesis reactor that improves temperature homogeneity within the reactor as a means to improve yield and catalyst lifetime.

The structured material being one of a micro-channel reactor, a milli-channel reactor, a monolith, a high-thermal-conductivity catalyst packing, a fractal device, or the like.

### Example 6

A device and method using a structured material in the downstream synthesis reactor that improves temperature homogeneity within the reactor as a means to improve yield and catalyst lifetime.

The structured material being one of a micro-channel reactor, a milli-channel reactor, a monolith, a high-thermal-conductivity catalyst packing, a fractal device, or the like. The device and method having one or more of the following features:

Use of thinner-walled reactor tubes, reactor channels, reactor packings, or reactor shells for high-pressure synthesis reactions enabled by the smaller hydraulic diameter of the reactor internal wetted features.

Using outputs from the engine-reformer to provide heat, electricity, shaft power, or pneumatic/hydraulic pressure to the downstream structured synthesis process; particularly as an approach to overcome any additional pumping/compression requirements introduced by the structured material.

Using additive manufacturing to produce the structured material and/or load the catalyst into the reactor.

### Example 7

The use of high thermal conductivity (HTC) supports within catalytic, packed bed reactors for chemical reactions such as methanol synthesis from syngas mixtures.

These supports are composed of open cell structures that allow fluid to permeate through their void space, such as foams used in other applications, ranging from heat exchangers, air/oil separators, filters, and scrubbers.

These foam structures can be composed of solid ligaments and be made with materials that exhibit high thermal conductivity values, leading to high rates of heat transfer and more uniform heating or cooling of flowing gases. These heat transfer rates may depend on the material of the foam (e.g., alloy, oxide, ceramic, etc.) as well as physical parameters such as the void space, the number of pores per inch (PPI, i.e., pore density), ligament thickness, and the intimacy of contact between the foam and wall surfaces. These enhancements are observed when flowing even inert gases through these foam materials, in the absence of any reactions, and their extents vary depending on the foam material (e.g., aluminum oxide, iron oxide, ferrochrome) and other physical properties.

The benefits of these thermally conductive, supporting foams can be exploited in reaction environments, among others, where non-uniform heating within a packed-bed reactor often leads to localized heat variations that could degrade or deactivate catalytic materials, leading to higher risk of runaway reactions, or to limitations in attainable conversions, because of the constraints imposed by chemical equilibria. Implementing open-cell structures within modular reactors for the conversion of syngas to methanol can potentially lead to lower catalyst requirements, less frequent catalyst replacements, smaller reactor vessel sizes, and lower pressure operation and compression work and thus translate to smaller overall system footprints and to process designs that are inherently simpler and ultimately less expensive to build and operate. Metal foams are preferred to alternative HTC structures, such as fine nonwoven metal meshes, because the foams are easier to pack and more practical for commercial reactors.

Commercially available catalysts for industrial methanol synthesis (typically composed of CuO, ZnO, and Al₂O₃) are sold in the form of cylindrical pellets (approximately 6 mm diameter×4 mm). These catalyst materials can be crushed and sieved to retain aggregates exhibiting a narrow particle size range (e.g., 0.25 to 1.0 mm), suitable for loading into the pores of the HTC foam support.

### Example 8

In an embodiment of a reactor of the general type shown in FIG. 7, the height of the catalyst bed, within a 0.94″ I.D. custom-built stainless steel reactor, was kept constant (3-in.) between loadings with catalyst aggregates, crushed to the same particle size range (0.25 to 1.0 mm) and the packed HTC, which required 40-45 grams of total catalyst loading. (The reactor can use the HTC of Example 7.) In this example, the aluminum 6101 HTC foam consists of pores exhibiting an average diameter (1.27 mm; 20 ppi) sufficient for accommodating these aggregates foam, and a relative density of 8-10%. A 1/16″ O.D. stainless steel thermowell was positioned at the centerline of the catalyst bed and extended along the length of the reactor, in order to measure the temperature, using a K-type thermocouple (0.02-in. diameter), at different axial positions (x) along the total length (L) of the bed.

Methanol synthesis reactions were performed at given pressures (up to 50 bar) and temperatures (200 to 250° C.) by flowing syngas mixtures over freely-loaded catalyst aggregates or aggregates packed in the HTC-support, at variable gas hourly space velocities (GHSV, units of (L/h)_{inlet gas}/L_{catalyst bed}). The reactor was enclosed within a heated oven furnace, the temperature of which (Tₒᵥₑₙ) was kept constant; at all bed positions, Tₒᵥₑₙ was lower than the reactor temperature (Tᵣₓₜᵣ), reflecting the heat generated from the exothermic methanol formation from CO and CO₂ hydrogenation. Temperatures were measured at varying axial positions within and upstream of the catalyst bed.

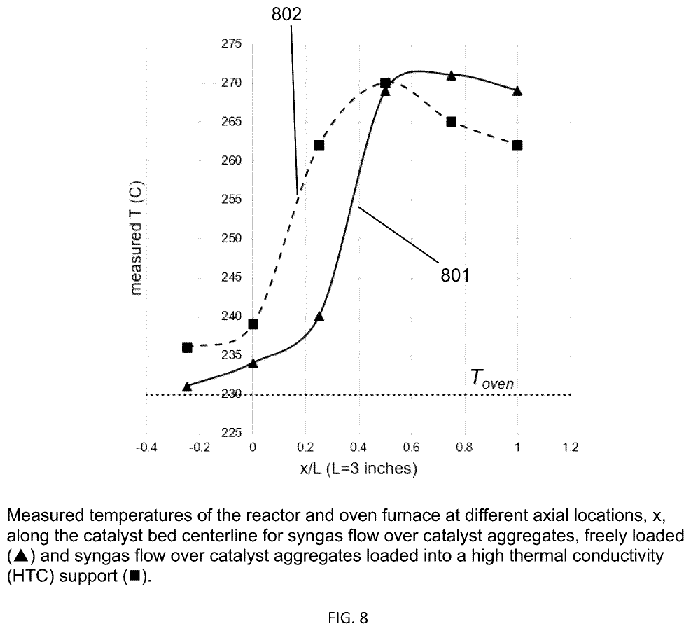

Less variability in temperature, along the catalyst bed, was evident for the HTC-packed system, relative to freely loaded catalyst aggregates, particularly after the first 25% of the bed volume (i.e., x/L from 0.25 to 1.0), as observed in FIG. 8. These temperature profiles reflect the distribution of heat caused by both reaction exotherms and heat transfer phenomena within the bed and the reactor wall surfaces, with the more uniform temperature profile observed on the HTC-packed system, representing a more rapid distribution of reaction exotherms across a greater reactor (axial) distance. Rapid and sufficient preheating of gas lines upstream of the reactor vessel (and within the oven) was confirmed by temperature measurements during inert N₂ gas flow. Improved temperature uniformity during reaction with the HTC-packed system can be seen in the graph of FIG. 8, which shows the measured temperatures of the reactor and oven furnace at different axial locations, x, along the catalyst bed centerline for syngas flow over catalyst aggregates freely loaded line 801 and syngas flow over catalyst aggregates loaded into a high thermal conductivity (HTC) support dashed line 802.

In general, for such methanol synthesis reactions, higher temperatures near the inlet of the bed facilitate reaction kinetics while lower temperatures near the outlet of the bed promote higher equilibrium conversion levels for exothermic reactions; both conditions are met by the use of these HTC-supports, as evidenced in FIG. 8. The greater temperature uniformity garnered from the use of HTC-supports thus provides each molecule with a more similar processing experience, leading to the higher H₂ and carbon conversions observed at all GHSV and an intrinsically more active catalyst bed for the HTC-support system, as shown in Table 1.

TABLE 1
Catalytic performance differences for methanol synthesis reactions on Cu/Zn/Al₂O₃
between catalyst aggregates loaded freely (If) and packed in a high thermal
conductivity support (HTC).

| GHSV (h⁻¹) | T_{outlet, HTC} - T_{outlet, If} (° C.)* | H₂ conv._{.HTC}/H₂ conv._{If} | C conv._{HTC}/C conv._{If} |
|---|---|---|---|
| 2000 | −5 | 1.07 | 1.08 |
| 9000 | −14 | 1.32 | 1.35 |
| 18750 | −11 | 1.28 | 1.30 |

*Tₒᵤₜₗₑₜ is the temperature measured at the reactor outlet, i.e., x/L = 1.0
HTC = aggregates loaded into a high thermal conductivity support
If = aggregates loaded freely

### Example 9

The use of high thermal conductivity (HTC) supports within catalytic, packed bed reactors for chemical reactions such as methanol synthesis from syngas mixtures. The device and method having one or more of the following features:

The methanol reactor being a multi-tubular boiling water reactor with HTC supports and catalyst in the tubes and boiling water on the shell side.

The methanol reactor being a quench reactor with HTC supports and catalyst in one or more substantially adiabatic sections of the reactor vessel with quench gas introduced between the sections.

The HTC supports being made of materials with high thermal conductivity such as copper, aluminum, graphite, or zinc.

The reformer reactor being an engine reformer.

### Example 10

The use of high thermal conductivity (HTC) supports within catalytic, packed bed reactors for chemical reactions such as deoxygenation (deoxo) of syngas mixtures or catalytic oxidation of tail gas.

### Example 11

The use of high thermal conductivity (HTC) supports within catalytic, packed bed reactors for chemical reactions as part of a system that includes one or more of the following features:

Reactive separation (e.g., reactive distillation, reactive adsorption, reactive absorption, reactive membrane separation) of products or byproducts.

Non-thermal separation (e.g., sonoseparation, membrane separation) of products or byproducts.

Application of mass-produced equipment (e.g., internal combustion engines) as low-cost reactors.

### Example 12

The embodiments of the present inventions, including the Examples are used to improve and enhance the systems and methods disclosed and taught in US Patent Publication No. 2022/0388842, the entire disclosure of which is incorporated herein.

It is noted that there is no requirement to provide or address the theory underlying the novel and groundbreaking advantages, performance or other beneficial features and properties that are the subject of, or associated with, embodiments of the present inventions. Nevertheless, various theories are provided in this specification to further advance the art in this important area, and in particular in the important area of hydrocarbon exploration, production and downstream conversion. These theories put forth in this specification, and unless expressly stated otherwise, in no way limit, restrict or narrow the scope of protection to be afforded the claimed inventions. These theories many not be required or practiced to utilize the present inventions. It is further understood that the present inventions may lead to new, and heretofore unknown theories to explain the conductivities, fractures, drainages, resource production, chemistries, and function-features of embodiments of the methods, articles, materials, devices and system of the present inventions; and such later developed theories shall not limit the scope of protection afforded the present inventions.

The various embodiments of devices, systems, activities, methods and operations set forth in this specification may be used with, in or by, various processes, industries and operations, in addition to those embodiments of the Figures and disclosed in this specification. The various embodiments of devices, systems, methods, activities, and operations set forth in this specification may be used with: other processes industries and operations that may be developed in the future; with existing processes industries and operations, which may be modified, in-part, based on the teachings of this specification; and with other types of gas recovery and valorization systems and methods. Further, the various embodiments of devices, systems, activities, methods and operations set forth in this specification may be used with each other in different and various combinations. Thus, for example, the configurations provided in the various embodiments of this specification may be used with each other. For example, the components of an embodiment having A, A′ and B and the components of an embodiment having A″, C and D can be used with each other in various combination, e.g., A, C, D, and A, A″, C and D, etc., in accordance with the teaching of this specification. Thus, the scope of protection afforded to the present inventions should not be limited to a particular embodiment, configuration or arrangement that is set forth in a particular embodiment, example, or in an embodiment in a particular Figure.

The invention may be embodied in other forms than those specifically disclosed herein without departing from its spirit or essential characteristics. The described embodiments are to be considered in all respects only as illustrative and not restrictive.

1. A small-scale, low capital intensity (CI) plant for converting a syngas into a higher-value product, the plant comprising:
a. a reactor unit configured to receive a flow of a syngas; wherein the reactor unit is configured to convert the syngas into a liquid product;
b. wherein the reactor unit is a small-scale processing unit; and,
c. wherein the reactor unit has a low CI.

2. The plant of claim 1, further comprising:
a. an air inlet for receiving a flow of air;
b. a flare gas inlet for receiving a flow of a flare gas;
c. a reformer in fluid communication with the air inlet and flare gas inlet; wherein the reformer is configured to receive the flows of the flare gas and air; and,
d. the reformer configured to convert the air and flare gas into the syngas, and thereby provide the flow of the syngas to the reactor.

3. The plant of claim 1, wherein the reactor unit is a two-stage unit.

4. The plant of claim 1, wherein the reactor unit comprises a means for reactive separation.

5. The plant of claim 1, wherein the reactor unit comprises a means for reactive separation, wherein the means for reactive separation comprises one or more of a reactive adsorption device, a reactive distillation device, and a reactive membrane device.

6. The plant of claim 1, wherein the reactor unit comprises a micro-channel reactor.

7. The plant of claim 1, wherein the reactor unit comprises a milli-channel reactor.

8. The plant of claim 1, wherein the reactor unit comprises a structured catalyst.

9. The plant of claim 1, wherein the reactor unit comprises a high-thermal-conductivity catalyst packing.

10. The plant of claim 1, wherein the reactor unit comprises a, fractal device.

11. The plant of claim 1, wherein the CI is less than about $110,000/bpd.

12. The plant of claim 1, wherein the CI is from about $110,000/bpd to $45,000/bpd.

13. The plant of claim 1, wherein the capacity is less than about 1,000 bpd.

14. The plant of claim 1, wherein the capacity is from about 2 bpd to 900 bpd.

15. The plant of claim 1, where in the liquid product comprises methanol.

16. The plant of claim 1, where in the liquid product consists of refined grade methanol.

17. The plant of claim 1, wherein the liquid product comprises ammonia.

18. The plant of claim 1, wherein the liquid product consists essentially of ammonia.

19. A small-scale, low capital intensity (CI) plant for converting a flare gas into methanol, the plant comprising:
a. an air inlet for receiving a flow of air;
b. a flare gas inlet for receiving a flow of a flare gas;
c. a reformer in fluid communication with the air inlet and the flare gas inlet; wherein the reformer is configured to receive the flows of the flare gas and air;
d. the reformer in fluid communication with a reactor unit; wherein the reactor unit is configured to receive a flow of the syngas from the reformer; and wherein the reactor unit is configured to convert the syngas into methanol;
e. the reactor unit is configured to conduct a reactive separation process;
f. wherein the reactor unit is a small-scale processing unit.

20. The plant of claim 19, wherein the reactive separation process comprises a sweep.

21. The plant of claim 20, wherein the sweep comprises a liquid sweep.

22. The plant of claim 20, wherein the sweep comprises a gaseous sweep.

23. The plant of claim 19, wherein the reactive separation process comprises a reactive adsorption.

24. The plant of claim 19, wherein the reactive separation process comprises a reactive distillation.

25. The plant of claim 19, wherein the reactive separation process comprises a reactive membrane separation.

26. The plant of claim 19, comprising a methanol refining unit.

27. A small-scale, low capital intensity (CI) plant for converting a flare gas into methanol, the plant comprising:
a. an air inlet for receiving a flow of air;
b. a flare gas inlet for receiving a flow of a flare gas;
c. a reformer in fluid communication with the air inlet and the flare gas inlet; wherein the reformer is configured to receive the flows of the flare gas and air;
d. the reformer in fluid communication with a reactor unit; wherein the reactor unit is configured to receive a flow of the syngas from the reformer; and wherein the reactor unit is configured to convert the syngas into methanol; and,
e. the reactor unit comprising a sonoseparator;
f. wherein the reactor unit is a small-scale processing unit.

28. A small-scale, low capital intensity (CI) plant for converting a flare gas into methanol, the plant comprising:
a. an air inlet for receiving a flow of air;
b. a flare gas inlet for receiving a flow of a flare gas;
c. a reformer in fluid communication with the air inlet and the flare gas inlet; wherein the reformer is configured to receive the flows of the flare gas and air;
d. the reformer in fluid communication with a reactor unit; wherein the reactor unit is configured to receive a flow of the syngas from the reformer; and wherein the reactor unit is configured to convert the syngas into methanol; and,
e. the reactor unit comprising a microchannel reactor;
f. wherein the reactor unit is a small-scale processing unit.

29. The plant of claim 28, wherein the microchannel reactor comprises a plurality of cooling plates and a plurality of reaction plates.

30. The plant of claim 28, wherein the microchannel reactor comprises a reaction plate with sweep.

31. A small-scale, low capital intensity (CI) plant for converting a flare gas into methanol, the plant comprising:
a. an air inlet for receiving a flow of air;
b. a flare gas inlet for receiving a flow of a flare gas;
c. a reformer in fluid communication with the air inlet and the flare gas inlet; wherein the reformer is configured to receive the flows of the flare gas and air;
d. the reformer in fluid communication with a reactor unit; wherein the reactor unit is configured to receive a flow of the syngas from the reformer; and wherein the reactor unit is configured to convert the syngas into methanol; and,
e. the reactor unit comprising a high thermal conductivity (HTC) catalyst bed;
f. wherein the reactor unit is a small-scale processing unit.

32. The plant of claim 31, wherein the HTC bed comprises catalyst aggregates loaded into a metal foam support.

33. The plant of claim 31, wherein the catalyst support is aluminum.

34. The plant of claim 31, wherein the relative density of the foam is less than 10%.

35. The plant of claim 31, wherein the methanol is refined grade methanol.

36. The plant of claim 31, wherein the CI is less than about $110,000/bpd.

37. The plant of claim 31, wherein the CI is from about $45,000/bpd to $110,000/bpd.

38. The plant of claim 31, wherein the capacity is less than about 1,000 bpd.

39. The plant of claim 31, wherein the capacity is from about 2 bpd to 900 bdp.

40. The plant of claim 31, wherein the plant is an onsite plant and located adjacent to a source of flare gas.

41. The plant of claim 40, wherein the source of flare gas is an oil well.

42. The plant of claim 40, wherein the reactor unit comprises a catalytic bed reactor having a high thermal conductivity support.

43. A method of onsite conversion of a flare gas to a liquid product using a small-scale, low capital intensity (CI) plant, the method comprising:
a. receiving a flow of a flare gas from a flare gas source;
b. providing the flare gas flow to a reformer engine;
c. converting the flare gas flow in the reformer engine into a syngas, thereby providing a syngas flow;
d. providing the syngas flow to a reactor unit;
e. processing the syngas into a liquid product in the reactor unit, wherein the processing comprises a reactive separation process.

44. The method of claim 43, wherein the reactor unit comprises a first reactor and a second reactor, and the processing is a two-stage process using the first reactor and the second reactor, and wherein the reactive separation process takes place in the second reactor.

45. The method of claim 43, wherein the reactive separation process comprises a reactive adsorption.

46. The method of claim 43, wherein the reactive separation process comprises a reactive distillation.

47. The method of claim 43, wherein the reactive separation process comprises a reactive membrane separation.

48. The method of claim 43, wherein the processing comprises using a catalytic bed reactor having a high thermal conductivity support.

49. The method of claim 43, wherein the processing comprises using a microchannel reactor.

50. The method of claim 43, further comprising a sonoseperation process to purify the liquid product.

51. The method of claim 43, wherein the source of the flare gas is an oil field and the method is carried out at the oil field.

52. The method of claim 43, wherein the CI is less than about $110,000/bpd.

53. The method of claim 43, wherein the CI is from about $45,000/bpd to $110,000/bpd.

54. The method of claim 43, wherein less than about 1,000 bpd of liquid product is produced.

55. The method of claim 43, wherein from about 2 bpd to 900 bdp of liquid product is produced.

56. The method of claim 43, where in the liquid product comprises methanol.

57. The method of claim 43, where in the liquid product consists of refined grade methanol.

58. The method of claim 43, wherein the liquid product comprises ammonia.

59. The method of claim 43, wherein the liquid product consists essentially of ammonia.

60. The method of operating the plant of claim 1.

61. A method of designing a small-scale, low capital intensity (CI) plant for converting a flare gas into methanol, the method comprising:
a. selecting components of a reactor unit to conduct a syngas to methanol process;
b. wherein the components of the reactor unit include components to conduct a reactive separation process;
c. optimizing the syngas to methanol process, the reactive separation process, or both, to provide a design for reactor unit having a small-scale and a low CI.

62. The method of claim 61, wherein the reactor unit has a CI of less than about $110,000/bpd.

63. The method of claim 61, wherein the reactor unit has a CI from about $45,000/bpd to $110,000/bpd.

64. The method of claim 61, wherein the reactor unit has a capacity of less than about 1,000 bpd.

65. The method of claim 61, wherein the reactor unit has a capacity from about 2 bpd to 900 bdp.

66. The method of 61, wherein the plant is configured to provide a refined grade methanol.
